(19) United States
(12) Patent Application Publication
(10) Pub. No.: US 2023/0404520 A1
(43) Pub. Date: Dec. 21, 2023

(54) METHODS AND SYSTEMS FOR PHOTOACOUSTIC COMPUTED TOMOGRAPHY OF BLOOD FLOW

(71) Applicant: California Institute of Technology, Pasadena, CA (US)

(72) Inventors: Yang Zhang, Pasadena, CA (US); Joshua Olick-Gibson, Pasadena, CA (US); Lihong Wang, Arcadia, CA (US)

(21) Appl. No.: 18/336,834

(22) Filed: Jun. 16, 2023

Related U.S. Application Data
(60) Provisional application No. 63/352,887, filed on Jun. 16, 2022.

(51) Int. Cl. A61B 8/06; A61B 8/08

(52) U.S. Cl. CPC A61B 8/065; A61B 8/5269

(57) ABSTRACT

Photoacoustic computed tomography of blood flow methods and systems that reconstruct photoacoustic images, use a spatiotemporal filter to extract at least one blood component from the images, and estimate blood flow measurements from the at least one blood component.

### CROSS-REFERENCES TO RELATED APPLICATIONS

This application claims priority to and the benefit of U.S. Provisional Patent Application No. 63/352,887, titled “FUNCTIONAL PHOTOACOUSTIC COMPUTED TOMOGRAPHY OF BLOOD FLOW,” filed on Jul. 16, 2022, which is hereby incorporated by reference in its entirety and for all purposes.

### FEDERALLY SPONSORED RESEARCH OR DEVELOPMENT

This invention was made with government support under Grant No(s). EB029823, NS102213, CA220436 awarded by the National Institutes of Health. The government has certain rights in the invention.

### FIELD

Certain aspects relate generally to the field of medical imaging, and more specifically, to photoacoustic computed tomography of blood flow that can be employed, e.g., in in-vivo hemodynamic imaging.

### BACKGROUND

Accurate blood flow measurement provides invaluable functional information about physiological processes in healthy and diseased tissue. Some existing techniques that can take blood flow measurements include magnetic resonance imaging (MRI) and Doppler ultrasound. MRI can suffer, however, from low temporal resolution. On other hand, Doppler ultrasound relies on an axial component of blood flow to calculate flow speed from frequency shifts, which makes the method less sensitive to transverse flow. Optical modalities such as laser Doppler imaging (LDI) and optical coherence tomography (OCT) provide contrast to endogenous chromophores, however, these techniques suffer from shallow depth penetration due to strong ballistic light attenuation in biological tissue.

Photoacoustic Tomography (PAT) is an imaging technique that can combine rich optical contrast with ultrasonic detection to enable fast, high-resolution, deep tissue imaging of endogenous chromophores, such as hemoglobin. An example of a PAT method can be found in Wang, L. V., “Tutorial on Photoacoustic Microscopy and Computed Tomography,” IEEE Journal of Selected Topics in Quantum Electronics,” (2008). PAT has advantages over MRI due to its high temporal resolution and low-cost, and, unlike ultrasound, PAT can distinguish between oxygenated and deoxygenated blood. Furthermore, due to its ultrasonic detection, PAT can image at greater depths than pure optical modalities. As such, PAT is uniquely positioned as a functional, anatomical, and molecular imaging modality which can simultaneously measure hemoglobin oxygen saturation (sO2) and blood flow. When combined, these two metrics can provide crucial physiological information regarding hallmarks of cancer, such as angiogenesis and hypermetabolism as discussed in Yao, J., Maslov, K., Wang, L. V., “In vivo Photoacoustic Tomography of Total Blood Flow and Potential Imaging of Cancer Angiogenesis and Hypermetabolism,” Technology in Cancer Research & Treatment (2012).

PAT has two main forms of implementation: Optical Resolution Photoacoustic Microscopy (OR-PAM) and Photoacoustic Computed Tomography (PACT). While both techniques can measure sO2, thus far, only OR-PAM has been demonstrated to measure in-vivo blood flow as discussed in Yao, J., Wang, L., Yang, J. M. et al., “High-speed label-free functional photoacoustic microscopy of mouse brain in action, Nature Methods, (2015). OR-PAM uses point-by-point scanning of an optical focus to enable transverse spatial resolution on the order of microns as discussed in Li, L., Li, Y., Zhang, Y. et al., “Snapshot photoacoustic topography through an ergodic relay of optical absorption in vivo,” Nature Protocols,” (2021). As such, OR-PAM has been used to measure blood flow by tracking individual red blood cells (RBCs), whose diameters range from 7-8 microns as discussed in Kinnunen, M., Kauppila, A., Karmenyan, A., & Myllylä, R., “Effect of the size and shape of a red blood cell on elastic light scattering properties at the single-cell level,” Biomedical optics express (2011). However, this measurement is limited to the optical diffusion limit in biological tissue (≤1 mm) due to the inability to focus light beyond one transport mean free path as discussed in Wang, L V and Beare, G. K., “Photoacoustic Tomography: Ultrasonically Breaking through the Optical Diffusion Limit,” Optics in the Life Sciences (2011).

Certain conventional PACT techniques use wide-field illumination coupled with an array of ultrasonic detectors to image vasculature at the expense of a poorer spatial resolution than with OR-PAM as can be found, e.g., in Yao, J, Wang, L. V., “Photoacoustic brain imaging: from microscopic to macroscopic scales,” Neurophotonics (2014). Generally speaking, however, conventional PACT techniques are not able to measure blood flow in deep tissue because 1) as compared to OR-PAM, these conventional PACT techniques have a lower resolution, preventing them from being able to resolve individual RBCs; and 2) the photoacoustic signal within the lumen of a vessel is suppressed relative to the signals at the boundaries due to the random summation of absorption signals from millions of RBCs in each lumen imaging voxel as can be found in Guo, Z., Li L., Wang L.V., “On the speckle-free nature of photoacoustic tomography,” Medical Physics (2009). Previous work by Brunker et al. has found that whole blood PACT velocity measurement in a phantom is possible for high center frequency (f_{c}) probes (f_{c}>30 MHz), as discussed in Brunker, J., & Beard, P. (2016), “Velocity measurements in whole blood using acoustic resolution photoacoustic Doppler,” Biomedical optics express (2016), but this center frequency constraint limits the depth capability of in-vivo flow measurement. Furthermore, while recent efforts discussed in Pakdaman Zangabad, R., Iskander-Rizk, S., van der Meulen, P., et al., “Photoacoustic flow velocity imaging based on complex field decorrelation,” Photoacoustics (2021) utilize PACT speckle field decorrelation to extract velocity measurements in ink phantoms and chicken embryos, direct imaging of blood flow beyond the optical diffusion limit has remained elusive.

### SUMMARY

Certain aspects relate generally to the field of medical imaging, and more specifically, to photoacoustic computed tomography of blood flow.

Certain aspects pertain to a photoacoustic computed tomography method that includes reconstructing a sequence of photoacoustic images based acoustic data detected by an ultrasonic transducer array, using a spatiotemporal filter to extract at least one blood component from each photoacoustic image, and estimating blood flow measurements from the at least one blood component.

Certain aspects pertain to a non-transitory computer readable media for generating one or more blood flow maps from acoustic data detected by an ultrasonic transducer array, the non-transitory computer readable media, when read by one or more processors, is configured to perform one or more operations. The one or more operations comprise reconstructing a sequence of photoacoustic images based acoustic data detected by an ultrasonic transducer array, using a spatiotemporal filter to extract at least one blood component from each photoacoustic image, and estimating blood flow measurements from the at least one blood component.

Certain aspects pertain to a photoacoustic computed tomography system comprising one or more light sources, an optical system for propagating light from the one or more light sources, an ultrasonic transducer array having an axis, and a computing system. The computing system is configured to execute instructions to reconstruct a sequence of photoacoustic images based on acoustic data detected by the ultrasonic transducer array, use a spatiotemporal filter to extract at least one blood component from each photoacoustic image to generate blood component images, and estimate one or more blood flow measurements from the blood component images.

These and other features are described in more detail below with reference to the associated drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of components of a photoacoustic computed tomography of blood flow system (PACTBF system), according to various embodiments.

FIG. 2 is a schematic diagram of components of a PACTBF system showing exemplary operations of PACTBF method, according to certain implementations.

FIG. 3 is a schematic diagram of components of a PACTBF system with a laser light source and an optional scanning mechanism, according to certain implementations.

FIG. 4 depicts a table comparing PACTBF techniques to ultrasound and OCT imaging techniques.

FIG. 5 is a flowchart depicting operations of a PACTBF method, according to an implementation.

FIG. 6 is a flowchart depicting operations of a PACTBF method for generating one or more vector flow maps, according to an implementation.

FIG. 7 is a flowchart depicting operations of a PACTBF method for generating one or more color Doppler maps, according to an implementation.

FIG. 8 is a flowchart depicting operations of a PACTBF method for generating one or more power Doppler maps, according to an implementation.

FIG. 9 depicts examples of PACTBF and ultrasound structural images and vector flow map images for two blood vessels, according to an implementation.

FIG. 10 depicts examples of PACTBF photoacoustic images and flow speed maps, according to an implementation.

FIG. 11 includes a PACTBF structural image and vector flow image during baseline, during cuff inflation, and at cuff release, according to an implementation.

FIG. 12 depicts examples of blood velocity speed change measurements generated by a PACTBF method, according to an implementation.

FIG. 13 depicts an example of a PACTBF color Doppler flow map, according to an implementation.

FIG. 14 depicts an example of a PACTBF power Doppler flow map, according to an implementation.

The figures and components therein may not be drawn to scale.

### DETAILED DESCRIPTION

Different aspects are described below with reference to the accompanying drawings. The features illustrated in the drawings may not be to scale. In the following description, numerous specific details are set forth in order to provide a thorough understanding of the presented implementations. The disclosed implementations may be practiced without one or more of these specific details. In other instances, well-known operations have not been described in detail to avoid unnecessarily obscuring the disclosed implementations. While the disclosed implementations will be described in conjunction with the specific implementations, it will be understood that it is not intended to limit the disclosed implementations.

Certain embodiments pertain to methods and systems for photoacoustic computed tomography of blood flow (PACTBF) methods and systems that can take blood flow measurements at depths beyond the optical diffusion limit of 1 mm. These PACTBF techniques may enable non-invasive imaging of deep blood vessels for mapping hemodynamics.

Certain embodiments pertain to PACTBF methods and systems that can quantify speed and direction of blood flow. In some cases, PACTBF techniques can use successive single-shot, photoacoustic images to directly visualize frame-to-frame propagation of the blood with a pixel-wise flow velocity estimation procedure. PACTBF techniques may enable hemodynamic quantification of deep veins at five times the optical diffusion limit (more than five millimeters), leading to vector mapping of blood flow in humans. These techniques may be useful monitoring and diagnosing vascular diseases and mapping circulatory system function.

Certain embodiments pertain to PACTBF methods and systems that use a spatiotemporal filter to extract blood components from successive single-shot photoacoustic images and estimate frame-to-frame propagation of the blood with a flow velocity estimation procedure. The flow velocity estimation procedure may determine blood flow measurements from the extracted blood components such as, e.g., velocity of blood flow, direction of blood flow and change in velocity. The flow velocity estimation procedure may also generate one or more blood flow maps such as a vector map of blood flow, a color Doppler map, and/or a power Doppler map.

### I. Photoacoustic Computed Tomography of Blood Flow (PACTBF) Systems

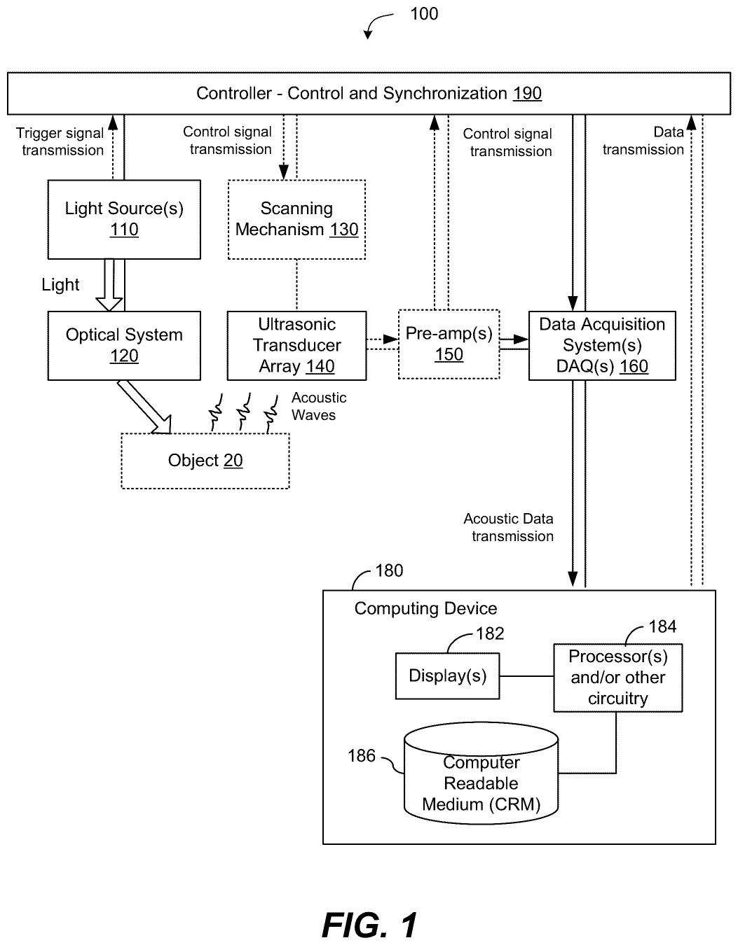

FIG. 1 is a block diagram of components of a system for photoacoustic computed tomography of blood flow (PACTBF system) showing signal flow, according to various embodiments. The PACTBF system 100 includes one or more light sources 110 (e.g., pulsed laser(s), continuous laser(s), or high power LED(s)) and an optical system 120 in optical communication with one or more light sources 110 to provide light. The optical system 120 includes one or more optical elements configured or configurable to propagate and/or alter light from one or more light sources 110 to an object 20 being imaged during operation. For example, the optical system 120 may include a fiber optic bundle that is optically coupled to one or more light sources 110 and that can be positioned to deliver light to object 20 during operation. At the instant in time illustrated in FIG. 1, object 20 is shown at a location at which it can receive light from optical system 120 during image acquisition. It would be understood that PACTBF system 100 does not require object 20 be included, e.g., at other instances.

The PACTBF system 100 also includes an ultrasonic transducer array 140, such as, e.g., an ultrasonic probe, for sampling acoustic waves. During operation, light from the one or more light sources 110 is delivered to object 20, which causes photoacoustic absorbers in object 20 to generate acoustic waves through thermoelastic expansion. Ultrasonic transducer array 140 is acoustically coupled with object 20 to detect the acoustic waves. In the example of an ultrasonic probe, the ultrasonic probe can be placed directly on, for example, a skin surface to sample acoustic waves from photoacoustic absorbers beneath the surface such as within the lumen of a blood vessel. In some cases, the object 20 may be placed in a tank of acoustic medium such as water. Ultrasonic transducer array 140 records one or more photoacoustic signals based on acoustic waves received. The transducer elements in the ultrasonic transducer array 140 may be arranged along a shape according to various implementations such as, e.g., in a linear array, a full-ring (circular) array, a hemispherical array, an arc-shaped array, a two-dimensional rectangular array, etc.

Optionally (denoted by dashed line), PACTBF system 100 includes one or more pre-amplifiers 150 for increasing amplitude (boosting) of photoacoustic signals received from the ultrasonic transducer array 140 and one or more data acquisition systems (DAQs) 160 for digitizing photoacoustic signals received from optional one or more pre-amplifiers 150 to generate digitized acoustic data. The optional pre-amplifier(s) 150 is shown in electrical communication (directly or via other circuitry) with ultrasonic transducer array 140 to receive the photoacoustic signals from the ultrasonic transducer array 140. The DAQ(s) 160 are shown in electrical communication (directly or via other circuitry) with optional pre-amplifier(s) 150 to receive the photoacoustic signals from pre-amplifier(s) 150. The DAQ(s) 160 may include at least one digitizer for digitizing photoacoustic signals. In implementations without pre-amplifier(s) 150, DAQ(s) 160 are in communication with the ultrasonic transducer array 140.

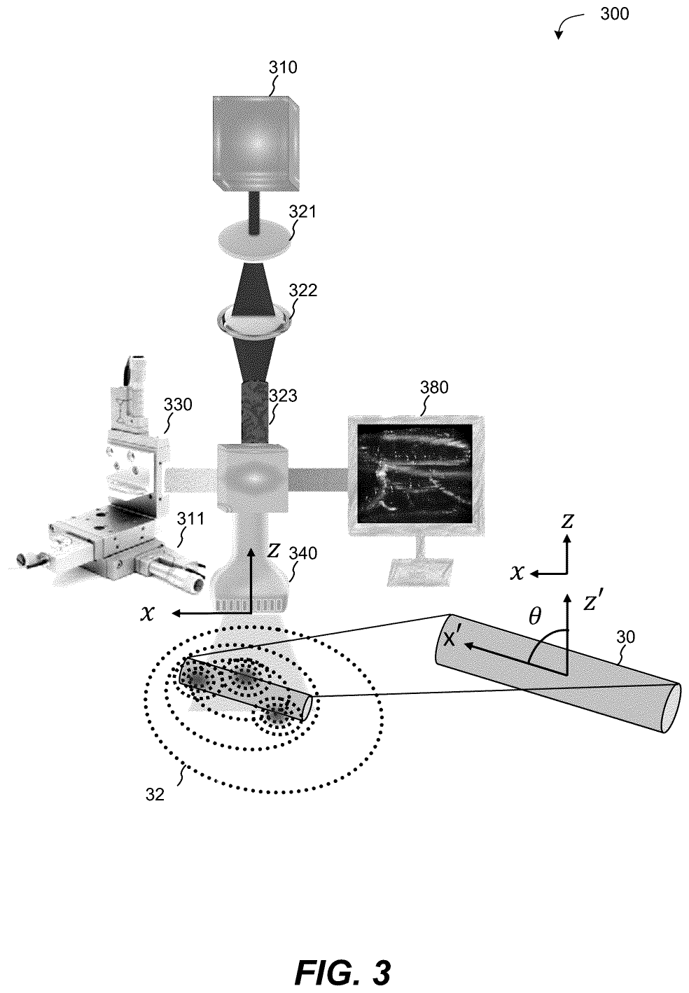

Optionally (denoted by dotted line), PACTBF system 100 may also include a scanning mechanism 130 for three-dimensional imaging implementations. The scanning mechanism 130 is coupled to or otherwise operably connected to ultrasonic transducer array 140 to be able to move (translate and/or rotate) the ultrasonic transducer array 140 to one or more positions during image acquisition. For example, the scanning mechanism 130 may be able to move ultrasonic transducer array 140 to a plurality of positions along an axis (e.g., elevational positions y₁, y₂, etc. along a y-axis such as shown in FIG. 3 for example) and hold the ultrasonic transducer array 140 at each position for a period of time. Some examples of time periods that may be used include about (e.g., +−1%) 10 seconds, about 15 seconds, and about 20 seconds. In one case, the time period may be in a range of about 10 seconds to about 20 seconds. An example of a scanning mechanism for linear movement is a linear stage KR4610D made by THK America, Inc.

PACTBF system 100 also includes a computing device 180 having one or more processors and/or other circuitry 184, an optional display 182 in electrical communication with the processor(s) 184, and a computer readable media (CRM) 186 in electronic communication with the processor(s) or other circuitry 184. Computing device 180 is in electronic communication with DAQ(s) 160 to receive acoustic data. Processor(s) and/or other circuitry 184 are in electrical communication with CRM 186 to store and/or retrieve data such as the acoustic data. The one or more processor(s) and/or other circuitry 184 are in electrical communication with optional display 586 for, e.g., displaying images. Although not shown, computing device 180 may also include a user input device for receiving data from an operator of PACTBF system 100. The computing device 180 may be, for example, a personal computer, an embedded computer, a single board computer (e.g., Raspberry Pi or similar), a portable computation device (e.g., tablet), or any other computation device or system of devices capable of performing the functions described herein.

One or more processors and/or other circuitry 184 execute instructions stored on the CRM 186 to perform one or more operations of a PACTBF method such as described in Section II. Processor(s) 184 and/or other circuitry 184 may execute instructions for: 1) communicating control signals to one or more components of PACTBF system 10, 2) performing image reconstruction to reconstruct a sequence of successive photoacoustic images using acoustic data, and/or 3) performing a flow estimation procedure to determine blood flow measurements and generate one or more flow maps from the blood flow measurements. For example, processor(s) and/or other circuitry 184 and/or one or more external processors may execute instructions that communicate control signals to scanning mechanism 130 to scan ultrasonic transducer array 140 along an axis between to two elevations (3D mode) or hold the ultrasonic transducer array 140 at a single elevation (2D mode) and send control signals to DAQ(s) 160 to synchronize recording photoacoustic signals received by ultrasonic transducer array 140. The computer readable media (CRM) 186 may be, e.g., a non-transitory computer readable media.

The PACTBF system 100 also includes a controller 190 for sending control signals to one or more components of PACTBF system 100 for control and synchronization. Controller 190 may include one or more processors and other circuitry and computer readable media. Controller 190 is in electronic communication with one or more light sources 110 to receive or send trigger signals. Controller 190 is also in electronic communication with DAQ(s) 660 to send control signals. Optionally (denoted by dashed line), controller 190 is in electronic communication with one or more pre-amplifiers 150 to send control signal(s), e.g., to adjust amplification. Optionally, controller 190 may also be in electronic communication with scanning mechanism 130 to send control signals to control the movement and/or hold positions of ultrasonic transducer array 140. For example, in one implementation, to synchronize components of PACTBF system 10, one or more light sources 110 are configured to transmit a trigger signal to controller 190 that triggers transmission of control signals to the DAQ(s) 160 to record photoacoustic signals and to scanning mechanism 130 to move ultrasonic transducer array 140 to synchronize the recording of photoacoustic signals at different positions (e.g., axial positions) of ultrasonic transducer array 140. Optionally, computing device 180 may be in communication with controller 190 to send control signals with control and synchronization data. Computing device 180 is also in communication with DAQ(s) 160 to receive acoustic data.

The electrical communication between components of a PACTBF system may be in wired and/or wireless form. One or more of the electrical communications between components of a PACTBF system may be able to provide power in addition to communicate signals. During operation, digitized data may be first stored in an onboard buffer, and then transferred to the computing device of a PACTBF system, e.g., through a universal serial bus 2.0.

In some implementations, a PACTBF system includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

During operation, light from the one or more light sources 110 is delivered to object 20 causing photoacoustic absorbers to generate acoustic waves through thermoelastic expansion. Ultrasonic transducer array 140 is acoustically coupled to object 20 to detect the acoustic waves. For example, an acoustic medium such as an acoustic gel or water may be provided at least partially between the object 20 and ultrasonic transducer array 140. The acoustic data detected by the ultrasonic transducer array 140 may be streamed to computing device 180 by DAQ(s) 160. After which, computing device 180 may reconstruct a sequence of photoacoustic images and extract one or more blood components from the images using a spatiotemporal filter. Computing device 180 may also perform pixel-wise frame-by-frame tracking of the extracted blood components from the sequence of photoacoustic images (frames) to determine blood flow measurements such as a blood flow speed and direction of blood flow. Computing device 180 may also generate flow maps from the blood flow measurements such as a vector flow map, a color Doppler flow map, and/or Power Doppler flow map).

In certain implementations, the one or more light sources of a PACTBF system include one or more lasers (e.g., pulsed or continuous laser). In one aspect, the one or more light sources may include a tunable narrow-band pulsed laser such as, e.g., one of a quantum cascade laser, an interband cascade laser, an optical parametric oscillator, or other pulsed laser that can be tuned to different narrow bands (e.g., a near-infrared band). In another aspect, the one or more light sources may include a pulsed laser of a single wavelength or approximately a single wavelength. In another aspect, the one or more light sources may include multiple lasers of the same type. For example, multiple lasers of the same type may be used where each of the lasers has a lower power. In another aspect, the one or more light sources may include a combination of different types of lasers. For example, an optical parametric oscillator combined with an Nd:YAG laser may be used in one implementation. In other implementations, the one or more light sources may include a laser emitting diode (LED). For example, a 144 element LED array to irradiate a 50 mm×7 mm area at a frame rate of up to 500 Hz and at a depth of up to 5 mm can be employed such as the 144 element LED array described in Zhu, Y., Xu, G., Yuan, J. et al. Light Emitting Diodes based Photoacoustic Imaging and Potential Clinical Applications. Sci Rep 8, 9885 (2018).

In certain implementations, the one or more light sources include at least one pulsed laser with high pulse repetition frequencies (PRF) in a range from 10-100 Hz and with wavelengths in a range from 694-1064 nm. In one implementation, the one or more light sources include at least one pulsed laser with a PRF of at least 10 Hz. In another implementation, the one or more light sources include at least one pulsed laser having a PRF of about 100 Hz. In some implementations such as, e.g., implementation used for near-infrared light penetration, the optical wavelengths are at least 690 nm. In other implementations such as, e.g., implementations used for deep blood flow analysis, the optical wavelengths are at least 1064 nm. In some cases, the ultrasound probe center frequency is at least 5 MHz. For example, the ultrasound probe center frequency may be about 15 MHz according to one implementation.

The optical system of a PACTBF system includes one or more optical elements configured or configurable to propagate and/or alter light from the one or more light sources to the object being imaged during operation. Some examples of optical elements include lens(es), optical filter(s), a fiber bundle of one or more fiber optic strands, mirror(s), beam steering device(s), beam-splitter(s), optical fiber(s), relay(s), and/or beam combiner(s)). For example, the optical system may include a fiber optic bundle that is optically coupled to the one or more light sources and that that can be positioned to deliver light to an object being imaged.

In one aspect, the optical system is configured to convert a light beam from the one or more light sources into shaped illumination such as donut-shaped illumination (sometimes referred to herein as a “donut beam”). Donut-shaped illumination and other circular illumination may be generated by employing an engineered diffuser such as, e.g., an EDC15 diffuser made by RPC Photonics®. In one example, a PACTBF system includes an optic fiber bundle optically coupled to the one or more light sources for delivering light to the object being imaged. A donut beam can be used to circumferentially illuminate the specimen. For example, the optical system may include an axicon lens (e.g., an axicon lens having 25 mm diameter and a 1600 apex angle) followed by an engineered diffuser (e.g., EDC-10-A-2s made by RPC Photonics) to convert a light beam from the one or more light sources into a donut beam. The axicon lens may be positioned to receive a single laser beam propagated from a pulsed laser source and may be configured to convert the beam into a ring having a thickness and diameter and the engineered diffuser may be configured to expand the ring into a donut beam. The donut beam may be used to provide mass energy in homogenized, uniform illumination deep into tissue. An example of a technique for generating donut-shaped illumination can be found in U.S. patent application Ser. No. 16/464,958, titled “SINGLE-IMPULSE PANORAMIC PHOTOACOUSTIC COMPUTED TOMOGRAPHY (SIP-PACT),” and filed on Nov. 29, 2017, which is hereby incorporated by reference in its entirety.

The ultrasonic transducer array of a PACTBF system includes a plurality of N transducers (sometimes referred to herein as “transducer elements”) operable to collect photoacoustic signals, e.g., in parallel. Each transducer element has an aperture (e.g., a flat-rectangular aperture). The transducers have a height, a width, and a pitch. In one case, the pitch is about 1.35 mm. In one case, the width is 0.65 mm. In another case, the pitch is in a range of 1.20 mm to 1.50 mm. In another case, the height is about 5 mm. In another case, the height is in a range of 2 mm to 10 mm. In certain implementations, the ultrasonic transducer array is in the form of an ultrasonic probe having the plurality of transducers within a housing that can be placed directed on the surface of an object. For example, an ultrasonic probe can be placed directly on a skin surface. An example of an ultrasound probe that can be implemented includes high-frequency linear array probes at center frequencies of 15 MHz and 5 MHz with 256 and 128 channels, respectively.

The transducer elements in the ultrasonic transducer array may be arranged in various shapes according to different implementations such as, e.g., a linear array, a full-ring (circular) array, a hemispherical array, an arc-shaped array, a two-dimensional rectangular array, etc. In one implementation, a full-ring ultrasonic transducer array may be employed for panoramic detection. In one example, the full-ring ultrasonic transducer array (e.g., a 512-element full-ring ultrasonic transducer) includes transducer elements distributed along the circumference of a ring having a diameter and an inter-element spacing. The ring diameter may be at least 220 mm in one aspect, may be at least 200 mm in one aspect, or may be at least 250 mm in one aspect. In one aspect, the ring diameter is in a range of about 150 mm to about 400 mm. The inter-element spacing may be less than or equal to about 1.0 mm in one aspect, less than or equal to 0.7 mm in one aspect, less than or equal to 1.5 mm in one aspect, or less than or equal to 2.0 mm in one aspect. In one aspect, the inter-element spacing is in a range of 0 mm to about 5 mm.

The one or more DAQs of a PACTBF system record photoacoustic signals according to a sampling frequency. In one aspect, the sampling frequency is in a range from about 4 MHz to about 100 MHz. In another aspect, the sampling frequency is about 40 MHz. In some implementations, the one or more DAQs are configured to record photoacoustic signals after a short time delay, e.g., 100 s, after each light excitation such as a pulse laser pulse excitation.

In some implementations, the digitizers of one or more DAQs and any optional one or more pre-amplifiers of a PACTBF system are in one one-to-one mapped associations with the transducers in the ultrasonic transducer array. These one-to-one mapped associations allow for fully parallelized data acquisition of all ultrasonic transducer channels and avoids the need for multiplexing after each laser pulse excitation or other modulated or pulsed excitation illumination. With one-to-one mapped associations to the transducer elements, each ultrasound transducer element in the array is in electrical communication with one dedicated pre-amplifier channel (also referred to as “preamp channel”). The one dedicated pre-amplifier channel is configured to amplify only photoacoustic signals detected by the one associated/mapped ultrasound transducer. These one-to-one mapped associations between the transducers and the pre-amplifier channels allow for parallelized pre-amplification of the photoacoustic signals detected by the plurality of transducers in the ultrasound transducer array. With one-to-one mapped analog-to-digital sampling, each pre-amplifier is operatively coupled to a corresponding dedicated data channel of an analog-to-digital sampling device in a DAQ to enable parallelized analog-to-digital sampling of the plurality of pre-amplified PA signals. The pre-amplified PA signals produced by each individual preamp channel are received by a single dedicated data channel of the at least one analog-to-digital sampling devices. Any suitable number of pre-amplifier devices and/or DAQ devices may be used to provide the one-to-one mapping.

In implementations with one or more pre-amplifiers, each pre-amplifier may be set to a pre-amplifier gain determined by one or more factors. For example, the pre-amplifier gain may be determined based on one or more of a minimum signal-to-noise ratio and one or more operating parameters of the data acquisition and processing system components such as analog-to-digital sampling devices (digitizers) of the DAQs, signal amplifiers, buffers, and the computing device. In one aspect, the pre-amplifier gain is in a range that is high enough to enable transmission of the photoacoustic signals with minimal signal contamination, but below a gain that may saturate the dynamic ranges of the DAQ system used to digitize the photoacoustic signals amplified by the pre-amplifier(s).

In one implementation, a PACTBF system includes one or more pulsed lasers, an ultrasonic transducer array in the form of an ultrasound probe, a 256 channel DAQ, and a computing device. The one or more pulsed lasers have a high pulse repetition frequencies (PRF) in a range from 10-100 Hz and wavelengths in a range from 694-1064 nm. The ultrasound probe may be a high-frequency linear array probes at center frequencies of 15 MHz and 5 MHz with 256 and 128 channels, respectively. In one case, the ultrasonic probes are linked to the 256 channel DAQ module. The imaging data may be transferred to a host computer, e.g., via PCI express. In one implementation, the one or more light sources include at least one pulsed laser with a PRF of at least 10 Hz. In another implementation, the one or more light sources include at least one pulsed laser having a PRF of about 100 Hz. In some implementations such as, e.g., implementation used for near-infrared light penetration, the optical wavelengths are at least 690 nm. In other implementations such as, e.g., implementations used for deep blood flow analysis, the optical wavelengths are at least 1064 nm. In some cases, the ultrasound probe center frequency is at least 5 MHz. For example, the ultrasound probe center frequency may be about 15 MHz according to one implementation.

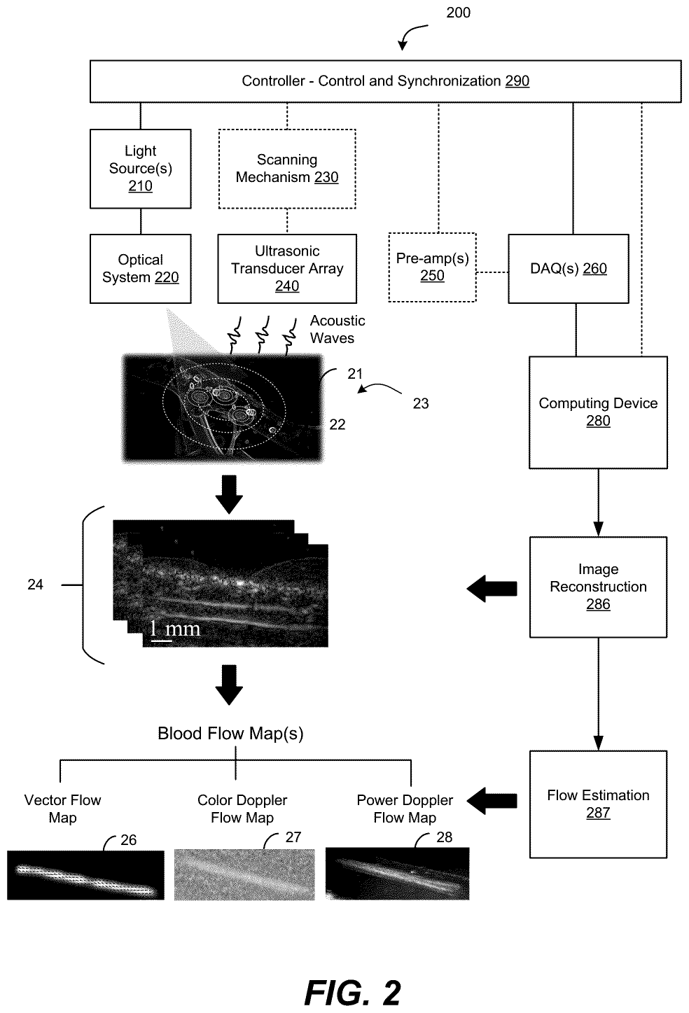

FIG. 2 is schematic diagram of components of a PACTBF system 200 and certain operations of a PACTBF method, according to various embodiments. The PACTBF system 200 includes one or more light sources 210 (e.g., pulsed laser, continuous laser, or high powered LED), an optical system 220 in optical communication with one or more light sources 210, and an ultrasonic transducer array 240, such as, e.g., an ultrasonic probe, for sampling acoustic waves. The illustrated example shows an example of a tissue 21 with a blood vessel 22 being illuminated by light from optical system 220, which causes photoacoustic absorbers in the lumen of blood vessel 22 to generate acoustic waves through thermoelastic expansion. The optical system 220 includes one or more optical elements configured or configurable to propagate and/or alter light from one or more light sources 210 to tissue 21. For example, optical system 220 may include a fiber optic bundle that is optically coupled to the one or more light sources 210 and that can be positioned to illuminate tissue 21.

Ultrasonic transducer array 240 is acoustically coupled with tissue 21 to be able to detect the acoustic waves generated by thermoelastic expansion. In the example of an ultrasonic probe, the ultrasonic probe may be placed directly on the surface of tissue 21 to sample acoustic waves from photoacoustic absorbers within the lumen of the blood vessel 22. Ultrasonic transducer array 240 records one or more photoacoustic signals based on acoustic waves detected. The transducer elements in the ultrasonic transducer array 240 may be arranged along a shape according to various implementations such as, e.g., in a linear array, a full-ring (circular) array, a hemispherical array, an arc-shaped array, a two-dimensional rectangular array, etc.

Optionally (denoted by dashed line), PACTBF system 200 also includes one or more pre-amplifiers 250 for increasing amplitude (boosting) of photoacoustic signals received from the ultrasonic transducer array 240. PACTBF system 200 also includes one or more data acquisition systems (DAQs) 260 for digitizing photoacoustic signals received from one or more pre-amplifiers 250 to generate digitized acoustic data. The one or more optional pre-amplifiers 250 are in electrical communication (directly or via other circuitry) with ultrasonic transducer array 240 to receive the photoacoustic signals. The one or more DAQs 260 have at least one digitizer and are in electrical communication (directly or via other circuitry) with one or more pre-amplifiers 250 to receive the boosted photoacoustic signals.

Optionally (denoted by dotted line), PACTBF system 200 may also include a scanning mechanism 230 for three-dimensional imaging implementations. The scanning mechanism 230 is coupled to or otherwise operably connected to ultrasonic transducer array 240 to be able to move (translate and/or rotate) the ultrasonic transducer array 240 to one or more positions during image acquisition. For example, the scanning mechanism 230 may be able to move ultrasonic transducer array 240 to a plurality of positions along an axis (e.g., elevational positions y₁, y₂, etc. along a y-axis such as shown in FIG. 3 for example) and hold the ultrasonic transducer array 240 at each position for a period of time (e.g., about (e.g., +−1%) 10 seconds, about 15 seconds, about 20 seconds, or in a range of about 10 seconds to about 20 seconds).

PACTBF system 200 also includes a computing device 280 having one or more processors and/or other circuitry and a computer readable media (CRM) in electronic communication with the processor(s) or other circuitry. Optionally, the computing device 280 may also have a display. Computing device 280 is in electronic communication with DAQ(s) 260 to receive acoustic data. The one or more processors and/or other circuitry execute instructions stored on the CRM to perform one or more operations of a PACTBF method. The one or more processors and/or other circuitry may execute instructions for: 1) communicating control signals to one or more components of PACTBF system 200, 2) performing image reconstruction to reconstruct one or more two-dimensional or three-dimensional photoacoustic images using acoustic data, 3) performing flow estimation to generate one or more blood flow maps. For example, processor(s) and/or other circuitry and/or one or more external processors may execute instructions that communicate control signals to scanning mechanism 230 to scan ultrasonic transducer array 240 along a y-axis between to two elevations (3D mode) or hold the ultrasonic transducer array 240 at a single elevation (2D mode) and send control signals to DAQ(s) 260 to synchronize recording photoacoustic signals received by ultrasonic transducer array 240. The computer readable media (CRM) 186 may be, e.g., a non-transitory computer readable media.

The PACTBF system 200 also includes a controller 290 for sending control signals to one or more components of PACTBF system 200 for control and synchronization. Controller 290 may include one or more processors and other circuitry and computer readable media. Controller 290 is in electronic communication with one or more light sources 210 to receive or send trigger signals. Controller 290 is also in electronic communication with DAQ(s) 660 to send control signals. Optionally (denoted by dashed line), controller 290 is in electronic communication with one or more pre-amplifiers 250 to send control signal(s), e.g., to adjust amplification. Optionally, controller 290 may also be in electronic communication with scanning mechanism 230 to send control signals to control the movement and/or hold positions of ultrasonic transducer array 240. For example, in one implementation, to synchronize components of PACTBF system 10, one or more light sources 210 are configured to transmit a trigger signal to controller 290 that triggers transmission of control signals to the DAQ(s) 260 to record photoacoustic signals and to scanning mechanism 230 to move ultrasonic transducer array 240 to synchronize the recording of photoacoustic signals at different positions (e.g., axial positions) of ultrasonic transducer array 240. Optionally, computing device 280 may be in communication with controller 290 to send control signals with control and synchronization data.

During operation, light from one or more light sources 210 is delivered to tissue 21 causing photoacoustic absorbers in the lumen of blood vessel 22 to generate acoustic waves through thermoelastic expansion. For example, optical system 220 may include a fiber optic bundle optically coupled to one or more light sources 210 that can be positioned to illuminate tissue 21. Ultrasonic transducer array 240, which is acoustically coupled to tissue 21, detects the acoustic waves. For example, ultrasonic transducer array 240 may be in the form of an ultrasonic probe that can be placed directly on the surface of tissue 21 to sample acoustic waves from photoacoustic absorbers within the lumen of blood vessel 22. The acoustic data detected by ultrasonic transducer array 240 may be streamed or otherwise transmitted to computing device 280 by DAQ(s) 260. After which, computing device 280 may perform operations of a PACTBF method including image reconstruction 286 to reconstruct a sequence (frames) of photoacoustic images 24 and flow estimation 287 to determine blood flow measurements such as one or more flow maps. Some examples of flow maps such as, e.g., a vector flow map, a color Doppler flow map, and/or a Power Doppler flow map. The flow estimation 287 may include, e.g., frame-by-frame tracking of lumen signals to determine blood flow speed and vector flow map of the hemodynamics.

FIG. 3 is schematic diagram of components of a PACTBF system 300 having a laser light source 310 (pulsed or continuous) and an optional scanning mechanism, according to one aspect. The PACTBF system 300 also includes an optical system with a diffuser 321 for diffusing the laser beam, a lens 322, and an optical fiber bundle 323. The illustrated example shows a blood vessel 30 being illuminated by light from optical system 320, which causes photoacoustic absorbers in the lumen of blood vessel 30 to generate acoustic waves 32 through thermoelastic expansion. PACTBF system 300 also includes an ultrasonic transducer array 340 in the form of an ultrasonic probe with a reference x-axis (azimuthal) and z-axis (axial). A y-axis (not shown) is perpendicular to a plane of the x-axis and the z-axis. The transducer elements in the ultrasonic transducer array 340 may be arranged in, e.g., a linear array, a full-ring (circular) array, a hemispherical array, an arc-shaped array, a two-dimensional rectangular array, etc.

Although not shown, the PACTBF system 300 also includes one or more data acquisition systems (DAQs) for digitizing photoacoustic signals detected by the ultrasonic transducer array 340 to generate digitized acoustic data. Optionally, PACTBF system 300 may also include one or more pre-amplifiers in electrical communication (directly or via other circuitry) with ultrasonic transducer array 340 to boost the photoacoustic signals. In one implementation, the components of PACTBF system 300 may be integrated onto a mobile platform.

PACTBF system 300 also includes an optional scanning mechanism 330 that may be employed in three-dimensional imaging implementations. The scanning mechanism 330 is coupled to or otherwise operably connected to ultrasonic transducer array 340 to be able to move (translate and/or rotate) the ultrasonic transducer array 340 to one or more positions during acquisition of a sequence (frames) of photoacoustic images. For example, the scanning mechanism 330 may be able to move ultrasonic transducer array 340 to a plurality of positions along the y-axis (e.g., elevational positions y₁, y₂, etc.).

PACTBF system 300 also includes a computing device 380 having one or more processors and/or other circuitry, a computer readable media (CRM) in electronic communication with the processor(s) or other circuitry, and an optional display. Computing device 380 is in electronic communication with one or more DAQ(s) 360 to receive acoustic data. The one or more processors and/or other circuitry execute instructions stored on the CRM to perform one or more operations of a PACTBF method. For example, the one or more processors and/or other circuitry may execute instructions for: 1) communicating control signals to one or more components of PACTBF system 300, 2) performing image reconstruction to reconstruct one or more two-dimensional or three-dimensional photoacoustic images using acoustic data, 3) performing flow estimation to generate one or more blood flow maps. Although not shown, PACTBF system 300 may also include a controller for sending control signals to one or more components of PACTBF system 300 for control and synchronization.

The ultrasonic probe is configured to take a series of single-shot, two-dimensional images. The PACTBF system 300 includes an optional scanning mechanism 330 (e.g., motorized translation stage) that can be employed to linearly scan the ultrasonic probe along its elevational direction along the y-axis to form three-dimensional (3D) images. The PACTBF system 300 may include a controller to synchronize the laser light source 310 and data acquisitions by the one or more DAQ(s) with the movement of the scanning mechanism 330.

During operation, the laser light source 310 delivers light to a biological tissue with the blood vessel 30 through the optical fiber bundle 323 that is coupled to the ultrasonic probe, which may be placed directly on the surface of the biological tissue. The light is absorbed by the hemoglobin molecules in blood vessel 30, and acoustic waves are generated through thermoelastic expansion. The acoustic waves are then detected by the ultrasonic probe and transmitted (e.g., streamed) from the one or more DAQ(s) to computing device 380 for image reconstruction using, e.g., the universal back-projection algorithm described in Xu, M., Wang L. V., “Universal back-projection algorithm for photoacoustic computed tomography,” (2005). The computing device 380 may also perform flow estimation to determine blood flow measurements such as one or more flow maps such as, e.g., a vector flow map, a color Doppler flow map, and/or a Power Doppler flow map. The flow estimation may include, e.g., frame-by-frame tracking of lumen signals to determine blood flow speed and vector flow map of the hemodynamics.

### II. Photoacoustic Computed Tomography of Blood Flow (PACTBF) Methods

According to certain implementations, PACTBF methods can measure blood flow beyond the optical diffusion limit up to 1 cm in depth. In some cases, these PACTBF methods use these blood flow measurements to determine flow speed and direction for different positions within the lumen of a blood vessel to generate one or more vector flow maps (images). These methods may detect variations in flow patterns throughout the lumen of the blood vessel. Additionally or alternatively, PACTBF methods can use these blood flow measurements to produce color Doppler velocity maps and/or power Doppler maps of the vascular lumen.

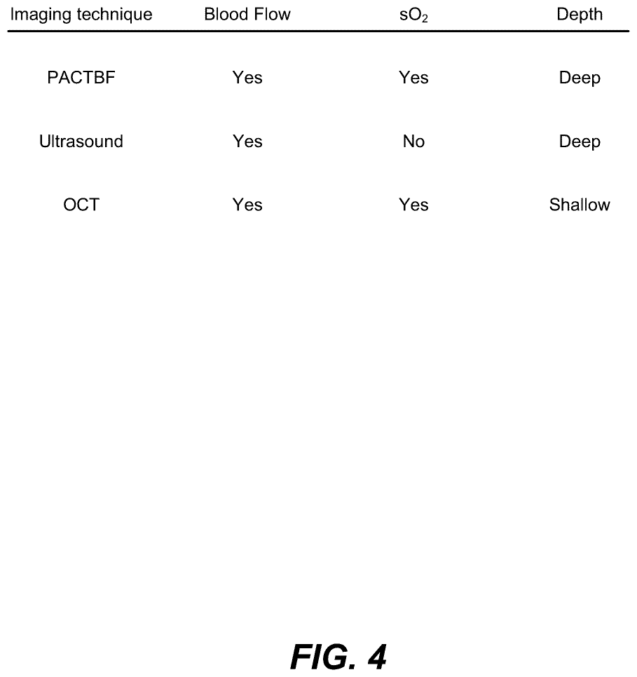

In certain embodiments, PACTBF methods can be used to directly measure both sO2 and blood flow functional changes in deep tissue. FIG. 4 depicts a table listing examples of advantages of PACTBF techniques of certain implementations over ultrasound and OCT imaging techniques. As shown, PACTBF techniques can image up to 1 cm, ultrasound can image a few centimeters deep, and OCT can image up to 1.2 mm. PACTBF techniques involve detecting the blood lumen signals relative to the blood boundary signals. PACTBF methods can image hand regions, arm regions, and other regions of a body.

FIG. 2 also depicts an example of an image reconstruction 286 operation and a flow estimation operation 287 of a PACTBF method, according to an implementation. In this example, the ultrasonic transducer array 240 may be in the form of an ultrasound probe that can be placed on top of the skin. The light source 210 may be a laser that can be activated to deliver light through a fiber optic bundle (e.g., fiber optic bundle 323 in FIG. 3) to a lumen region of the blood vessel 21. The hemoglobin content within red blood cells (RBCs) absorbs the light and converts it into heat. Based on the photoacoustic effect, acoustic waves are generated through thermoelastic expansion and detected by the ultrasound probe. The acoustic data may then be streamed to the computing device 280 through the DAQ(s) 260. At the image reconstruction operation 286, a universal back projection (UBP) process may be used to reconstruct the sequence of photoacoustic images 24 from the acoustic data. An example of a UBP process that can be used is described in Xu, M., Wang L. V., “Universal back-projection algorithm for photoacoustic computed tomography,” Physical Review (2005), which is hereby incorporated by reference for the details regarding the UBP reconstruction technique.

FIG. 2 also includes a photoacoustic computed tomography image 23 of an example of a blood vessel 21 located beyond 1 mm in depth. The illustrated photoacoustic computed tomography image 23 shows strong boundary signals and weaker lumen signals due to the boundary buildup effect. After acquiring multiple frames of acoustic data, a flow estimation operation 287 (e.g., a pixel-wise flow estimation) may be applied to the sequence of photoacoustic images 24 (video images) to extract blood flow measurements of blood flow inside the blood vessel 21 to generate blood flow maps such as a vector flow map, a color Doppler map, and/or a power Doppler map. For example, the flow estimation operation 287 may determine the direction and magnitude of blood flow inside the blood vessel 21 and generate a vector flow map. FIG. 2 includes an example of a vector flow map 26 that shows a laminar flow pattern in the lumen region that has a higher speed through the center of the blood vessel and a lower speed at the edges. FIG. 2 includes an example of a color Doppler flow map 27 and an example of a color Doppler flow map 28.

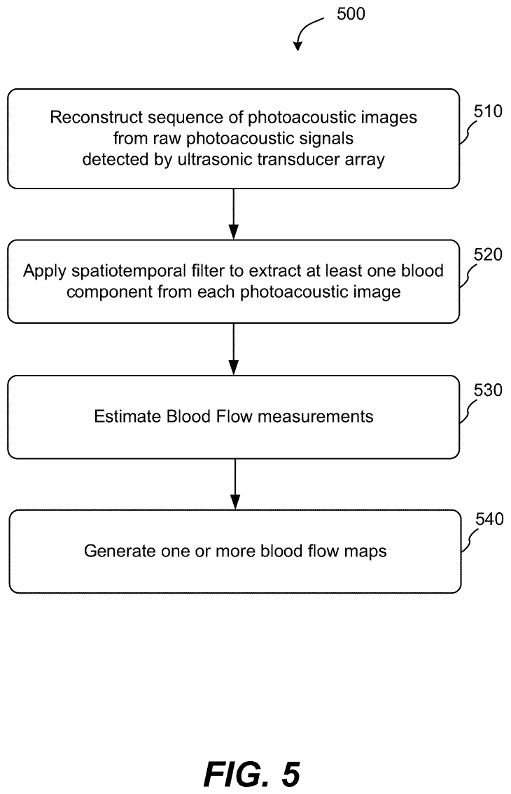

FIG. 5 is a flowchart depicting operations of a PACTBF method for generating one or more blood flow maps, according to various implementations. One or more of the operations may be performed by components of a PACTBF system such as PACTBF system 100, PACTBF system 200, or PACTBF system 300.

At operation 510, a sequence of photoacoustic images is reconstructed from raw photoacoustic signals detected by an ultrasonic transducer array of a PACTBF system. Image reconstruction may include (i) reconstructing one or more two-dimensional photoacoustic images and/or (ii) reconstructing a volumetric three-dimensional image for a volume scanned by the ultrasonic transducer array. Image reconstruction includes, at least in part, implementing an inverse reconstruction algorithm. Some examples of inverse reconstruction methods that can be used include: (i) forward-model-based iterative methods, (ii) time-reversal methods, and (iii) back projection methods. A 3D back projection method can be used to reconstruct a 3D volumetric image and a 2D back projection method can be used to reconstruct a 2D image. An example of a back projection method is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020, which is hereby incorporated by reference for this description. Another example of a back-projection method can be found in Anastasio, M. A. et al., “Half-time image reconstruction in thermoacoustic tomography,” IEEE Trans., Med. Imaging 24, pp 199-210 (2005). In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S patent application 2019/0307334, titled “SINGLE-IMPULSE PANORAMIC PHOTOACOUSTIC COMPUTED TOMOGRAPHY” and filed on May 29, 2019, which is hereby incorporated by reference in its entirety.

At operation 520, a spatiotemporal filter (e.g., a singular value decomposition (SVD)-based spatiotemporal filter) is applied to extract at least one blood component from the photoacoustic images. The spatiotemporal structure has three dimensions with two spatial axes (ultrasound probe azimuthal direction x and axial direction z) and one time axis (time t). The 3D dataset is reshaped into a 2D space-time matrix S_{structure}(x,z,t). A singular value decomposition (SVD) may be used to decompose the data matrix as follows:

S_{structure}(x,z,t)=Σ_{I=1}^{r}σᵢuᵢ(x,z)vᵢ^{T}(t), (Eqn. 1)
where r is the rank of the data matrix, σᵢ is the i^{th} singular value, T is the conjugate transpose, uᵢ(x,z) corresponds to the spatial domain, and vᵢ(t) corresponds to the temporal domain. An example of SVD decomposition is described in Demene, C. et al. Spatiotemporal Clutter Filtering of Ultrafast Ultrasound Data Highly Increases Doppler and fUltrasound Sensitivity. IEEE Trans. Med. Imaging 34, 2271-2285 (2015). The static or slow-moving components (i.e., tissue) correspond to the first few larger singular values and thus, the relatively fast-moving blood components can be extracted as:

S_{blood}(x,z,t)=S_{structure}(x,z,t)−Σᵢ₌₁^{λ}σᵢuᵢ(x,z)vᵢ^{T}(t). (Eqn. 2)
where λ is the cutoff of the singular values for extracting the blood component. Finally, the filtered 2D space-time matrix S_{blood}(x,z,t) is reshaped back to 3D with the same size as the original 3D dataset.

At operation 530, a flow estimation process is used to determine blood flow measurements (e.g., velocity, direction, change in velocity, mean intensity of images) in the sequence of photoacoustic images. At operation 540, a blood flow map is generated using the blood flow measurements.

### A. Vector Flow Map

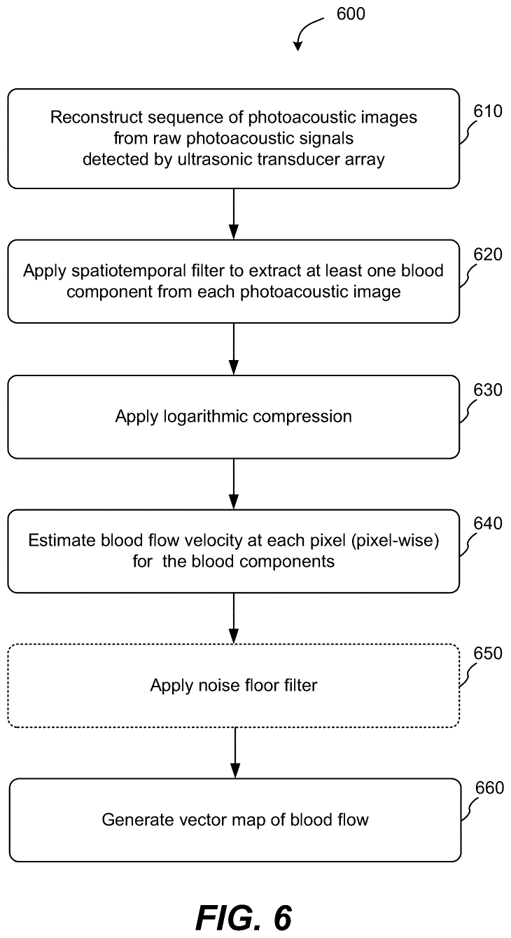

FIG. 6 is a flowchart depicting operations of a PACTBF method for generating one or more vector flow maps, according to implementations. These PACTBF methods may be able to measure blood flow to depths of more than 5 mm. One or more of the operations may be performed by components of a PACTBF system such as PACTBF system 100, PACTBF system 200, or PACTBF system 300.

At operation 610, a sequence of photoacoustic images is reconstructed from raw photoacoustic signals detected by an ultrasonic transducer array of a PACTBF system. Image reconstruction may include (i) reconstructing one or more two-dimensional photoacoustic images and/or (ii) reconstructing a volumetric three-dimensional image for a volume scanned by the ultrasonic transducer array. Image reconstruction includes, at least in part, implementing an inverse reconstruction algorithm. Some examples of inverse reconstruction methods that can be used include: (i) forward-model-based iterative methods, (ii) time-reversal methods, and (iii) back projection methods. A 3D back projection method can be used to reconstruct a 3D volumetric image and a 2D back projection method can be used to reconstruct a 2D image. In another aspect, a dual-speed-of sound (dual-SOS) photoacoustic reconstruction process may be used.

At optional (denoted by dashed line) operation 520, a spatiotemporal filter (e.g., a singular value decomposition (SVD)-based spatiotemporal filter) is applied to extract the blood component from the photoacoustic images. According to an implementation, a motion tracking procedure can be applied to the reconstructed images as provided in paragraph below.

The spatiotemporal structure has three dimensions with two spatial axes (ultrasound probe azimuthal direction x and axial direction z) and one time axis (time t). The 3D dataset is reshaped into a 2D space-time matrix S_{structure}(x,z,t). A singular value decomposition (SVD) may be used to decompose the data matrix as follows:

S_{structure}(x,z,t)=Σᵢ₌₁^{r}σᵢuᵢ(x,z)vᵢ^{T}(t), (Eqn. 3)
where r is the rank of the data matrix, σᵢ is the i^{th} singular value, T is the conjugate transpose, uᵢ(x,z) corresponds to the spatial domain, and vᵢ(t) corresponds to the temporal domain. An example of SVD decomposition is described in Demene, C. et al. Spatiotemporal Clutter Filtering of Ultrafast Ultrasound Data Highly Increases Doppler and Ultrasound Sensitivity. IEEE Trans. Med. Imaging 34, 2271-2285 (2015). The static or slow-moving components (i.e., tissue) correspond to the first few larger singular values and thus, the relatively fast-moving blood components can be extracted as:

S_{blood}(x,z,t)=S_{structure}(x,z,t)−Σᵢ₌₁^{λ}σᵢuᵢ(x,z)vᵢ^{T}(t). (Eqn. 4)
where λ is the cutoff of the singular values for extracting the blood component. Finally, the filtered 2D space-time matrix S_{blood}(x,z,t) is reshaped back to 3D with the same size as the original 3D dataset.

At operation 630, amplitude-based logarithmic compression is applied to filter the images to highlight the lumen signals. An example of an amplitude-based logarithmic compression procedure that can be employed can be found in Lee, Y., Kang, J. & Yoo, Y. Automatic dynamic range adjustment for ultrasound B-mode imaging. Ultrasonics 56, 435-443 (2015), which is hereby incorporated by reference for the amplitude-based logarithmic compression procedure. For amplitude-based logarithmic compression according to an implementation, a reconstructed image is normalized by the maximum value in the image, base-10 logarithm operation is applied to all values in the image, all values are multiplied by 20, and the images are displayed with a user-defined lower decibel cutoff.

A pixel-wise flow estimation method and noise floor filtering is employed to obtain the velocity of the blood flow. At operation 640, a pixel-wise flow estimation method is applied to determine a velocity at each pixel. For each pixel, a frame-to-frame velocity is estimated to form a 3D velocity structure with two spatial axes (e.g., ultrasound probe azimuthal direction x and axial direction z shown in FIG. 3) and one time axis (time t). According to an implementation, a motion tracking procedure can be applied to the reconstructed images as provided in paragraph below

In one implementation, the Farneback method for optical flow may be employed for pixel-wise flow estimation. The Farneback method approximates pixel neighborhoods as polynomial expansions. By assuming a constant intensity of the displaced neighborhoods between adjacent frames, the displacement field may be estimated. The pixel neighborhood in Frame 1 at position vector x may be approximated as:

f₁(x)=x^{T}A₁x+b₁^{T}x+c₁, (Eqn. 5)
in which the coefficients A₁ (a symmetric matrix), b₁ (a vector), and c₁ (a scalar) are estimated by a weighted least squares fit of the signal. The displaced pixel neighborhood in Frame 2 be approximated as:

$${f}_{2} ( x ) = {f}_{1} ( x - d ) = {( x - d )}^{T} {A}_{1} ( x - d ) + {b}_{1}^{T} ( x - d ) + {c}_{1} = {x}^{T} {A}_{1} x + {( {b}_{1} - 2 {A}_{1} d )}^{T} x + {d}^{T} {A}_{1} d - {b}_{1}^{T} d + {c}_{1} = {x}^{T} {A}_{2} x + {b}_{2}^{T} x + {c}_{2} , \qquad ( Eqn . 6 )$$

in which d is the displacement vector to be estimated. By equating the coefficients

A₁=A₂ (Eqn. 7)

b₂=b₁=−2A₁d (Eqn. 8)
we solve for d:

$$d = - \frac{1}{2} {A}_{1}^{- 1} ( {b}_{2} - {b}_{1} ) \qquad ( Eqn . 9 )$$

At optional (denoted by dashed line) operation 650, a noise floor filter is applied. The temporal standard deviation of each pixel's speed measurements is calculated to form a pixel-wise noise floor filter, below which frames with low speeds (i.e., measurements due to noise) are excluded. After acquiring a frame-to-frame velocity map at each time point, the temporal standard deviation of each pixel's speed is calculated. Before calculating the average velocity map at operation 660, for each pixel the speed values that fall below the standard deviation at that location are excluded.

At operation 660, a vector map of the blood flow is generated. For a given time period, the frame-to-frame velocity map at each time point is calculated. Optionally, each pixel's value can be average in the temporal domain to form the average velocity map in the given time period. The remaining frames (after noise floor filtering) are averaged across the temporal domain.

### B. Color Doppler Maps

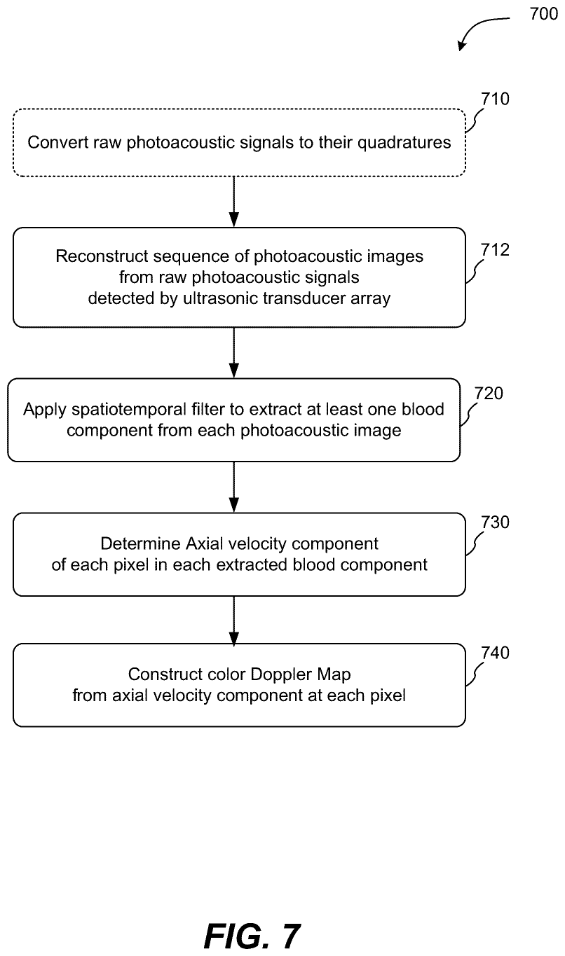

FIG. 7 is a flowchart depicting operations of a PACTBF method for generating one or more color Doppler maps, according to implementations. These PACTBF methods may be able to measure blood flow to depths of up to 1 cm. One or more of the operations may be performed by components of a PACTBF system such as PACTBF system 100, PACTBF system 200, or PACTBF system 300.

Optionally, at operation 710, the raw photoacoustic signals detected by an ultrasonic transducer array of a PACTBF system are converted to their quadratures, e.g., using the Hilbert transformation. If the signals are not converted to their quadrature form, the Doppler shift may be estimated in the frequency domain by taking a Fourier transform.

At operation 712, a sequence of photoacoustic images is reconstructed. Image reconstruction may include (i) reconstructing one or more two-dimensional photoacoustic images and/or (ii) reconstructing a volumetric three-dimensional image for a volume scanned by the ultrasonic transducer array. Image reconstruction includes, at least in part, implementing an inverse reconstruction algorithm. Some examples of inverse reconstruction algorithms that can be used include: (i) forward-model-based iterative methods, (ii) time-reversal methods, and (iii) back projection methods. A 3D back projection algorithm can be used to reconstruct a 3D volumetric image and a 2D back projection algorithm can be used to reconstruct a 2D image. An example of a back projection method that can be used is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020. Another example of a back-projection process can be found in Anastasio, M. A. et al., “Half-time image reconstruction in thermoacoustic tomography,” IEEE Trans., Med. Imaging 24, pp 199-210 (2005). In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S patent application 2019/0307334, titled “SINGLE-IMPULSE PANORAMIC PHOTOACOUSTIC COMPUTED TOMOGRAPHY” and filed on May 29, 2019.

At operation 720, a spatiotemporal filter (e.g., a singular value decomposition (SVD)-based spatiotemporal filter) is applied to extract the blood component from the photoacoustic images. The spatiotemporal structure dataset has three dimensions with two spatial axes (ultrasound probe azimuthal direction x and axial direction z) and one time axis (time t). The 3D dataset is reshaped into a 3D dataset into a 2D space-time matrix S_{structure}(x,z,t). A singular value decomposition (SVD) can be used to decompose the data matrix as follows:

S_{structure}(x,z,t)=Σᵢ₌₁^{r}λᵢuᵢ(x,z)vᵢ^{T}(t), (Eqn. 10)
where r is the rank of the data matrix, λᵢ is the i^{th} singular value, T is the conjugate transpose, uᵢ(x,z) corresponds to the spatial domain, and vᵢ(t) corresponds to the temporal domain. The static or slow-moving components (i.e., tissue) correspond to the first few larger singular values (i.e., the smallest singular value indices), whereas the noise components correspond to the last few smaller singular values (i.e., the largest singular value indices). Therefore, the relatively fast-moving blood components can be extracted as:

$${S}_{blood} ( x , z , t ) = {S}_{structure} ( x , z , t ) - {\sum }_{i = 1}^{{λ}_{l}} {λ}_{i} {u}_{i} ( x , z ) {v}_{i}^{T} ( t ) - {\sum }_{i = {λ}_{u}}^{r} {λ}_{i} {u}_{i} ( x , z ) {v}_{i}^{T} ( t ) = {\sum }_{i = {λ}_{l}}^{{λ}_{u}} {λ}_{i} {u}_{i} ( x , z ) {v}_{i}^{T} ( t ) \qquad ( Eqn . 11 )$$

where λᵢ and λᵤ are the lower and upper singular values cutoffs, respectively, for extracting the blood component. Finally, the filtered 2D space-time matrix S_{blood}(x,z,t) is reshaped back to 3D with the same size as the original 3D dataset.

At operation 730, the axial velocity component of each pixel in the extracted blood component is determined. Each pixel in an image contains an in-phase (I) and quadrature (Q) component and can be mathematically expressed in complex form as

S_{blood}(x,z,tₙ)=Iₙ+jQₙ=Aₙe^{jϕn}, (Eqn. 12)
where S_{blood}(x,z,tₙ) represents the blood component pixel value with coordinates (x,z) in the nth image frame, Aₙ=\sqrt{Iₙ²+Qₙ²} is the envelope of the signal, and ϕₙ=2πftₙ to is the phase of the received signal with frequency f at time tₙ. The mean Doppler frequency shift f̅ can then be estimated for each pixel from the mean phase shift Δ̅Ø̅ according to

$$\overline{f} ( x , z ) = \frac{\overline{Δ∅} ( x , z )}{2 π {T}_{P R F}} = \frac{1}{2 π {T}_{P R F} ( N - 1 )} {\sum }_{n = 1}^{N - 1} angle [ {S}_{b l o o d} ( x , z , {t}_{n + 1} ) {S}_{blood}^{*} ( x , z , {t}_{n} ) ] \qquad ( Eqn . 13 )$$

where N is the total number of frames, T^{PRF} is the time period associated with the pulse repetition frequency (PRF) of the laser, * denotes the complex conjugate, and the angle function computes the phase of a complex number z=x+jy as angle[z]=tan⁻¹[y/x]. Finally, the axial velocity component of each pixel can be obtained from

$${v}_{a xial} ( x , z ) = \frac{c \overline{f} ( x , z )}{{f}_{0}} \qquad ( Eqn . 14 )$$

where c is the speed of sound in the medium, and f₀ is the center frequency of the ultrasonic probe.

At operation 740, a color Doppler map is constructed from the axial velocity components determined at each pixel.

### C. Power Doppler Map

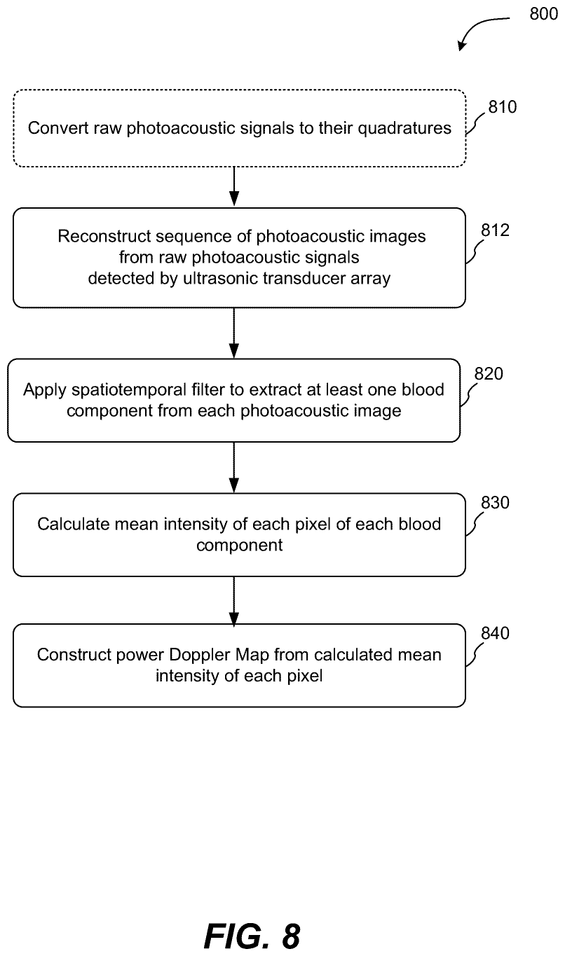

FIG. 8 is a flowchart depicting operations of a PACTBF method for generating one or more power Doppler maps, according to implementations. These PACTBF methods may be able to measure blood flow to depths of up to 1 cm. One or more of the operations may be performed by components of a PACTBF system such as PACTBF system 100, PACTBF system 200, or PACTBF system 300.

Optionally, at operation 810, the raw photoacoustic signals detected by an ultrasonic transducer array of a PACTBF system are converted to their quadratures, e.g., using the Hilbert transformation. If the signals are not converted to their quadrature form, the Doppler shift may be estimated in the frequency domain by taking a Fourier transform.

At operation 812, a sequence of photoacoustic images is reconstructed. Image reconstruction may include (i) reconstructing one or more two-dimensional photoacoustic images and/or (ii) reconstructing a volumetric three-dimensional image for a volume scanned by the ultrasonic transducer array. Image reconstruction includes, at least in part, implementing an inverse reconstruction algorithm. Some examples of inverse reconstruction algorithms that can be used include: (i) forward-model-based iterative methods, (ii) time-reversal methods, and (iii) back projection methods. A 3D back projection algorithm can be used to reconstruct a 3D volumetric image and a 2D back projection algorithm can be used to reconstruct a 2D image. An example of a back projection method that can be used is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020. Another example of a back-projection process can be found in Anastasio, M. A. et al., “Half-time image reconstruction in thermoacoustic tomography,” IEEE Trans., Med. Imaging 24, pp 199-210 (2005). In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S patent application 2019/0307334, titled “SINGLE-IMPULSE PANORAMIC PHOTOACOUSTIC COMPUTED TOMOGRAPHY” and filed on May 29, 2019.

At operation 820, a spatiotemporal filter (e.g., a singular value decomposition (SVD)-based spatiotemporal filter) is applied to extract the blood component from the photoacoustic images. The spatiotemporal structure dataset has three dimensions with two spatial axes (ultrasound probe azimuthal direction x and axial direction z) and one time axis (time t). The 3D dataset is reshaped into a 3D dataset into a 2D space-time matrix S_{structure}(x,z,t). A singular value decomposition (SVD) can be used to decompose the data matrix as follows:

S_{structure}(x,z,t)=Σᵢ₌₁^{r}λᵢuᵢ(x,z)vᵢ^{T}(t), (Eqn. 15)
where r is the rank of the data matrix, λᵢ is the i^{th} singular value, T is the conjugate transpose, uᵢ(x,z) corresponds to the spatial domain, and vᵢ(t) corresponds to the temporal domain. The static or slow-moving components (i.e., tissue) correspond to the first few larger singular values (i.e., the smallest singular value indices), whereas the noise components correspond to the last few smaller singular values (i.e., the largest singular value indices). Therefore, the relatively fast-moving blood components can be extracted as:

$${S}_{blood} ( x , z , t ) = {S}_{structure} ( x , z , t ) - {\sum }_{i = 1}^{{λ}_{l}} {λ}_{i} {u}_{i} ( x , z ) {v}_{i}^{T} ( t ) - {\sum }_{i = {λ}_{u}}^{r} {λ}_{i} {u}_{i} ( x , z ) {v}_{i}^{T} ( t ) = {\sum }_{i = {λ}_{l}}^{{λ}_{u}} {λ}_{i} {u}_{i} ( x , z ) {v}_{i}^{T} ( t ) , \qquad ( Eqn . 16 )$$

where λᵢ and λᵤ are the lower and upper singular values cutoffs, respectively, for extracting the blood component. Finally, the filtered 2D space-time matrix S_{blood}(x,z,t) is reshaped back to 3D with the same size as the original 3D dataset.

At operation 830, the mean intensity of the blood component images are calculated for each pixel according to:

$${I}_{P D} ( x , z ) = \frac{1}{N} {\sum }_{n = 1}^{N} {\vert {S}_{b l o o d} ( x , z , {t}_{n} ) \vert}^{2} \qquad ( Eqn . 17 )$$

where I_{PD} is the power Doppler value, which is proportional to the blood volume. Alternatively, the signal can be integrated in the frequency domain.

At operation 840, a power Doppler map is constructed from the mean intensity calculated at each pixel.

### III. Demonstration Examples

### A. Vector Flow Maps

According to certain implementations, PACTBF methods may be used for vector mapping of blood flow based on hemodynamic quantification of deep veins at up to five times the optical diffusion limit (more than five millimeters). By offering the capability for deep hemodynamic imaging with optical contrast, PACTBF techniques may be a powerful tool for monitoring and diagnosing vascular diseases and mapping circulatory system function.

An implementation of the PACTBF system 300 in FIG. 3 and the PACTBF method described in Section IA were used to acquire photoacoustic images of two blood vessels. The implementation of the PACTBF system 300 included an ultrasonic transducer array in the form of a linear array ultrasonic probe (f_{c}=15 MHz) coupled to the fiber bundle 323. The ultrasonic probe was placed on top of the skin of the human subject, and the laser light source 310 delivered light through the fiber bundle 323 to the blood vessels. The hemoglobin content within RBCs absorbs the light and converts it into heat. Based on the known photoacoustic effect, acoustic waves are generated through thermoelastic expansion and detected by the ultrasonic probe. The data was then streamed to the computing device 380 through the one or more DAQ(s), and universal back projection procedure was used to reconstruct the sequence of photoacoustic images. As a basis of comparison to other in-vivo speed measurements, Doppler ultrasound images were also taken of the same two blood vessels. In-vivo functional blood flow changes were also measured in response to a blood pressure cuff.

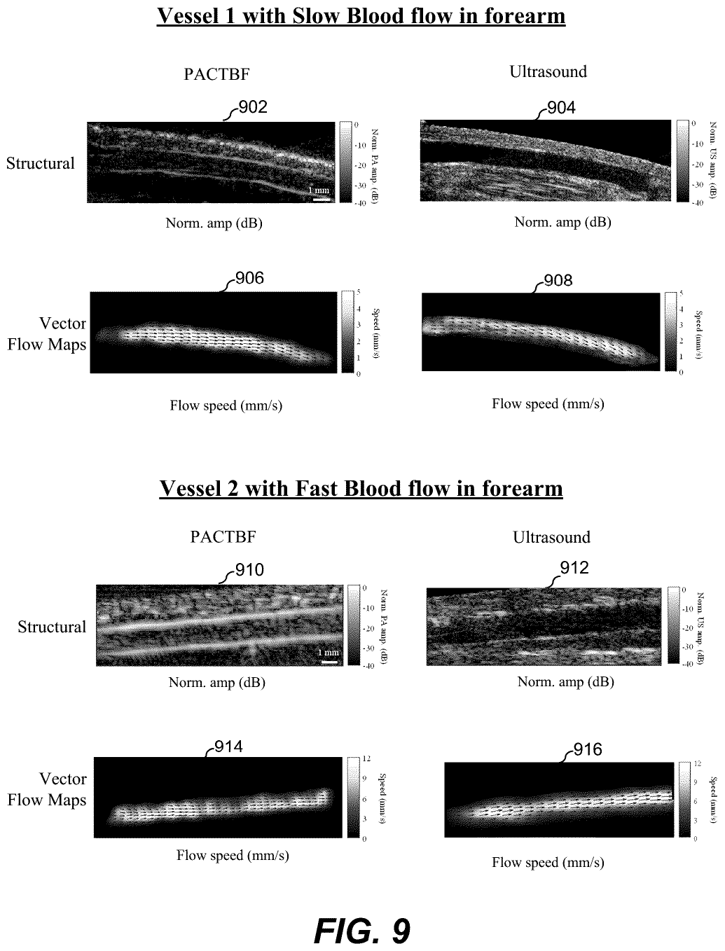

FIG. 9 shows (i) PACTBF structural photoacoustic images 902, 910 and ultrasound structural images 904, 912 for two blood vessels (Vessel 1 with slow blook flow and Vessel 2 with fast blood flow), and (ii) PACTBF vector flow maps 906, 914 and ultrafast ultrasound vector flow maps 908, 916.

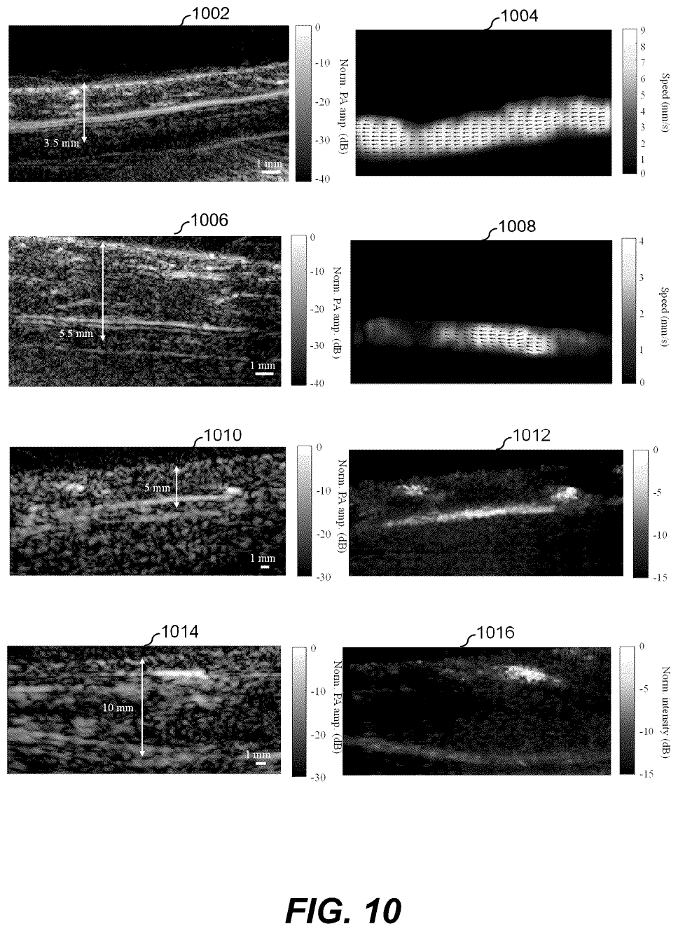

The PACTBF vector flow maps (imaging) may be obtained in blood vessels greater than 5 mm in depth. Moreover, vector flow imaging in PACTBF may be used to measure flow patterns at irregular interfaces in the blood vessel, such as valve regions. FIG. 10 depicts examples of photoacoustic images 1002, 1006, 1010, and 1014 and respective flow speed maps 1004, 1008, 1012, and 1016 from performing a PACTBF method, according to an implementation.

To evaluate functional changes, a subject's blood flow was measured before, during, and after inflating a blood pressure cuff. The cuff was placed against the brachial artery in the upper arm, and the imaged vessel was located in the metacarpal region, distal to the application site of the cuff. Fractional speed changes were measured relative to the baseline flow, which was acquired for approximately 8 seconds (800 frames at a 100 Hz laser pulse repetition frequency).

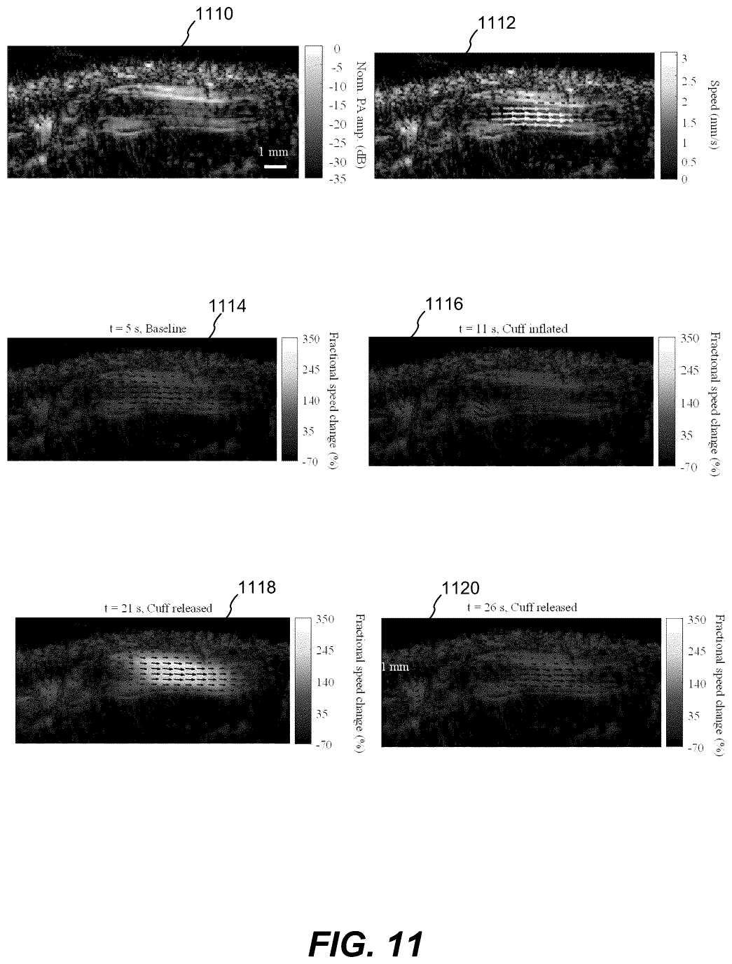

FIG. 11 includes a PACTBF structural image 1010 and vector flow image during baseline 1112, during cuff inflation 1112, and at cuff release 1114, according to an implementation. FIG. 11 also includes a PACTBF vector flow image at baseline flow 1014, PACTBF vector flow image during cuff inflation 1016, PACTBF vector flow image during release 1018, and PACTBF vector flow image after release 1020, according to an implementation.

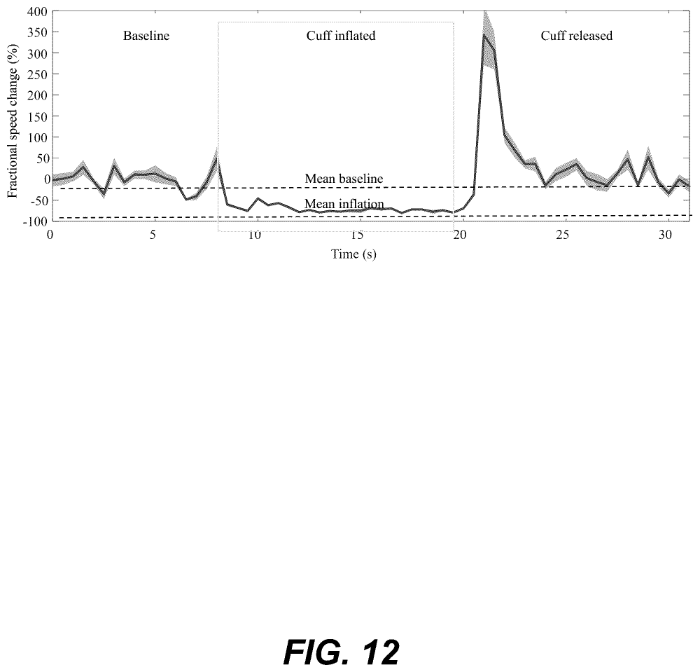

FIG. 12 is a graph of a plot of fractional speed change over time, according to an implementation. Fractional speed changes were measured relative to the mean baseline flow, which was acquired for approximately 8 seconds. The cuff inflation induced a flow speed decrease of approximately 70%, whereas the cuff release induced a transient flow speed increase of approximately 350% percent, followed by a return to the baseline. The plot of the functional speed change shows a flow speed decrease of approximately 70% while the cuff was inflated, followed by a transient spike of approximately 350% upon release of the cuff, and eventually a steady-state return to baseline flow. The 70% drop can be explained by incomplete cuffing, whereas the transient spike could be due to a pressure buildup induced by cuffing the brachial artery/vein pair, halting both the inlet and outlet of flow in the imaged vessel.

### B. Color and Power Doppler Flow Maps

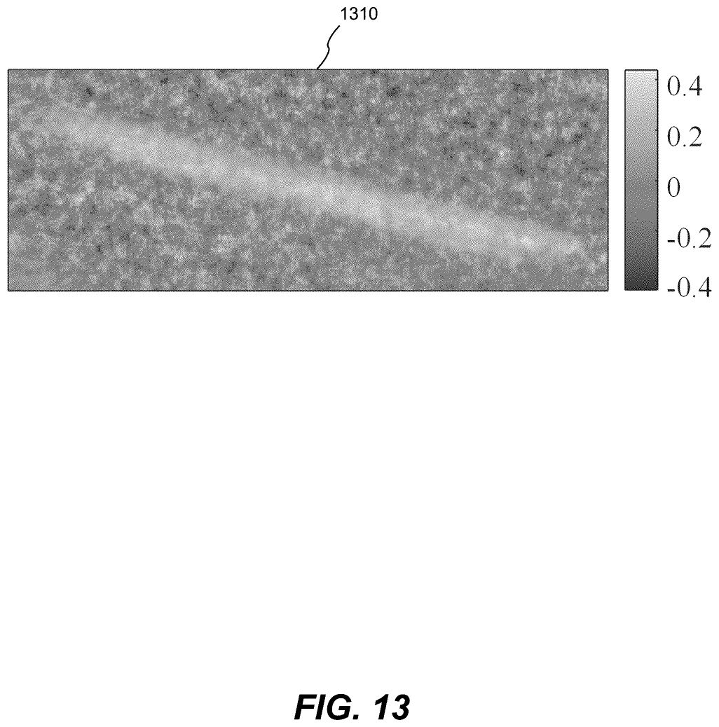

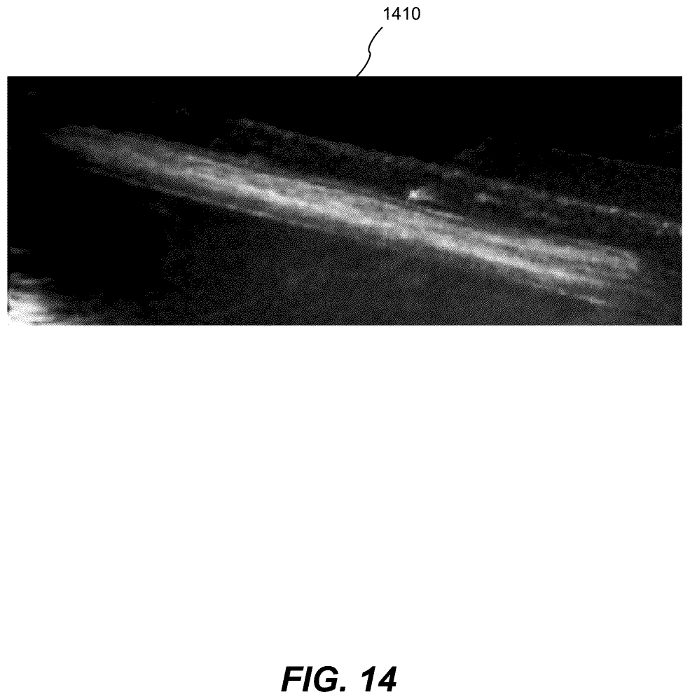

According to certain implementations, PACTBF methods may be used to generate color Doppler and power doppler flow maps based on hemodynamic quantification of deep veins at up to 1 cm. FIG. 13 depicts an example of a PACTBF color Doppler flow map, according to an implementation. FIG. 14 depicts an example of a PACTBF power Doppler flow map, according to an implementation.

An implementation of the PACTBF system 300 in FIG. 3 and the PACTBF method described in Section IA were used to acquire photoacoustic images. The implementation of the PACTBF system 300 included an ultrasonic transducer array in the form of a linear array ultrasonic probe (f_{c}=15 MHz) coupled to the fiber bundle 323. The ultrasonic probe was placed on top of the skin of the human subject, and the laser light source 310 delivered light through the fiber bundle 323 to the blood vessels. The hemoglobin content within RBCs absorbs the light and converts it into heat. Based on the known photoacoustic effect, acoustic waves are generated through thermoelastic expansion and detected by the ultrasonic probe. The data was then streamed to the computing device 380 through the one or more DAQ(s), and universal back projection procedure was used to reconstruct the sequence of photoacoustic images.

Modifications, additions, or omissions may be made to any of the above-described implementations without departing from the scope of the disclosure. Any of the implementations described above may include more, fewer, or other features without departing from the scope of the disclosure. Additionally, the steps of described features may be performed in any suitable order without departing from the scope of the disclosure. Also, one or more features from any implementation may be combined with one or more features of any other implementation without departing from the scope of the disclosure. The components of any implementation may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C #, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic medium such as a hard-drive or a floppy disk, or an optical medium such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed implementations have been described in some detail to facilitate understanding, the described implementations are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain implementations herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or implementations of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A photoacoustic computed tomography method, comprising:
reconstructing a sequence of photoacoustic images based on acoustic data detected by an ultrasonic transducer array;
using a spatiotemporal filter to extract at least one blood component from each photoacoustic image; and
estimating blood flow measurements from the at least one blood component.

2. The photoacoustic computed tomography method of claim 1, further comprising generating one or more blood flow maps from the blood flow measurements.

3. The photoacoustic computed tomography method of claim 2, wherein the one or more blood flow maps comprise one or more of a vector map, a color Doppler map, or a power Doppler map.

4. The photoacoustic computed tomography method of claim 2, wherein estimating blood flow measurements comprises:
applying logarithmic compression; and
estimating a blood velocity value at each pixel of the at least one blood component.

5. The photoacoustic computed tomography method of claim 4, further comprising applying a noise floor filter to remove one or more estimated blood velocity values.

6. The photoacoustic computed tomography method of claim 4, further comprising generating a vector map from estimated blood velocity values.

7. The photoacoustic computed tomography method of claim 2, wherein estimating blood flow measurements comprises determining an axial velocity at each pixel of the at least one blood component.

8. The photoacoustic computed tomography method of claim 7, further comprising constructing a color Doppler map from the axial velocity at each pixel of the at least one blood component.

9. The photoacoustic computed tomography method of claim 2, wherein estimating blood flow measurements comprises determining a mean intensity of each pixel of the at least one blood component.

10. The photoacoustic computed tomography method of claim 9, further comprising constructing a power Doppler map from the mean intensity of each pixel of the at least one blood component.

11. The photoacoustic computed tomography method of claim 1, wherein the at least one blood component is of a vessel being imaged at a depth of more than 5 mm.

12. The photoacoustic computed tomography method of claim 1, wherein the at least one blood component is of a vessel being imaged at a depth of up to 1 cm.

13. A non-transitory computer readable media for generating one or more blood flow maps from acoustic data detected by an ultrasonic transducer array, the non-transitory computer readable media, when read by one or more processors, is configured to perform one or more operations comprising:
reconstructing a sequence of photoacoustic images based acoustic data detected by the ultrasonic transducer array;
using a spatiotemporal filter to extract at least one blood component from each photoacoustic image; and
estimating blood flow measurements from the at least one blood component.

14. A photoacoustic computed tomography system, comprising:
one or more light sources;
an optical system for propagating light from the one or more light sources;
an ultrasonic transducer array having an axis; and
a computing system configured to execute instructions to:
reconstruct a sequence of photoacoustic images based on acoustic data detected by the ultrasonic transducer array;
use a spatiotemporal filter to extract at least one blood component from each photoacoustic image; and
estimate blood flow measurements from the at least one blood component.

15. The photoacoustic computed tomography system of claim 14, further comprising a scanning mechanism configured to scan the ultrasonic transducer array along the axis.

16. The photoacoustic computed tomography system of claim 14, wherein the computing system is further configured to generate one or more blood flow maps from the blood flow measurements estimated.

17. The photoacoustic computed tomography system of claim 16, wherein the one or more blood flow maps comprise one or more of a vector map, a color Doppler map, or a power Doppler map.
